(19) United States
(12) Patent Application Publication
(10) Pub. No.: US 2015/0375961 A1
(43) Pub. Date: Dec. 31, 2015

(54) MONITORED STAIRLIFT

(71) Applicant: ACORN MOBILITY SERVICES LIMITED, Yorkshire (GB)

(72) Inventor: John Stewart JAKES, MONACO (MC)

(73) Assignee: ACORN MOBILITY SERVICES LIMITED, Yorkshire (GB)

(21) Appl. No.: 14/764,851

(22) PCT Filed: Jan. 31, 2014
(86) PCT No.: PCT/GB2014/050279

§ 371 Date: Jul. 30, 2015

(30) Foreign Application Priority Data
Feb. 1, 2013 (GB) 1301871.8

(51) Int. Cl. B66B 5/00; A61B 5/00; B66B 9/08

(52) U.S. Cl. CPC B66B 5/0012; B66B 9/08; A61B 5/0015; B66B 2009/0876

(57) ABSTRACT

The invention provides a stairlift device which comprises passenger carrying means, guide means and transporting means, wherein the stairlift additionally comprises means for the detection of vital health data of passengers and transmittal of said data to remote locations. Typically, the passenger carrying means comprises a chair which is connected to the guide means by way of mounting means, and the guide means comprises a close fitting channel into which said mounting means may be inserted, and which defines the path of travel of said passenger carrying means. The stairlift device of the invention allows for the remote monitoring of the health of users, by sending vital health data to designated recipients, such as health professionals, carers and family members. The invention also provides a method for the detection of vital health data of passengers and transmittal of said data to remote locations using the stairlift device, and the use of the stairlift device for the detection of vital health data of users and transmittal of said data to remote locations.

### FIELD OF THE INVENTION

The present invention relates to stairlifts. In particular, the invention provides an improvement in the functioning of stairlifts which allows for the primary health indicators of individuals using the stairlifts to be monitored in real time, thereby providing significant advances in emergency response times and general health care.

### BACKGROUND TO THE INVENTION

The use of stairlifts has become widespread in recent years as a means of overcoming problems of immobility associated with infirmity or old age, which result in difficulties in climbing stairs. In larger buildings, such difficulties have long been overcome by the use of lifts or escalators but, until more recent years, no simple facilities were available which were useable in the smaller domestic environment. Consequently, the inability of certain personnel to climb or descend stairs in a safe manner without some form of assistance often proved to be a source of great inconvenience and handicap.

Consequently, stairlift devices have been developed which greatly simplify the task of ascending or descending stairs for the elderly and infirm. Generally, such devices comprise (a) passenger carrying means, which generally comprises seating means, wherein a person is seated for the purpose of ascending or descending the stairs, but may simply comprise means for carrying a standing passenger, such as a platform or similar attachment, (b) mounted guide means, by which the device is attached to the stairs and floor, and which defines the distance and direction of travel of the passenger carrying means, and (c) transporting means, which is operable to move the passenger carrying means along the guide means. Typically, in a simple arrangement, a rear attachment from, say, a chair would be movably located in the mounted guide means, which would possibly be in the form of a guide rail affixed to the stairs, and the chair would be caused to ascend or descend the stairs by driving means such as an electric motor, which would propel the chair along the guide means in the desired direction.

Not surprisingly, stairlifts have been found to be of great benefit in the field of health care and mobility and have significantly improved the quality of life for many people, and there is a large and growing market for these devices, especially amongst people in older age groups. In addition to problems of immobility, these older age groups typically also increasingly encounter a range of other health problems, which can include, for example, heart problems and diabetes. These particular health issues are prime examples of the type of health problems where medical professionals wish to more closely monitor the health of the sufferers on an ongoing basis, in order to improve care and treatment. Specifically, there is a requirement to facilitate the monitoring of health indicators from a remote location, thereby allowing for, typically, health professionals to have access to current data on a real-time basis, such that response times can be dramatically improved.

Devices which allow for health data to be monitored are known from the prior art. Thus, for example, US-A-2007/0022522 discloses a toilet seat which comprises a health monitoring system, including a seat body which includes a weighing sensor for measuring the weight of a user, an Inductor chip for detecting physical constitution on its upper surface, and a displaying and processing device in its front surface. The output signals from the weighing sensor and the inductor chip are input into the displaying and processing device.

U.S. Pat. No. 6,809,643 teaches a health monitoring system for use with a child car seat which has sensors mounted in the seat to monitor one or more health conditions of the seat's occupant. A processor monitors the sensor's signals and generates status signals related to the monitored conditions. A transmitter wirelessly transmits the status signals to a remotely located receiver and a signaling device coupled to the receiver produces at least one sensory (e.g., visual, audible, tactile) output based on the status signals.

EP-B-0558975 relates to a networked health care and monitoring system capable of providing updated information on the health condition of individuals which includes measuring devices associated with household appliances such as toilet systems which are adapted to monitor the vital information passively in response to the use thereof in connection with routine living activities of the individuals. The system may further include measuring devices associated with other household appliances, such as an ergometer having health care and maintenance functions and adapted to control the appliances based on the information monitored by the measuring devices in the system. The system may be arranged in the centralized network configuration wherein the measuring devices are connected via a local area network with a data controller where all the obtained information is stored.

With the objective of enabling remotely located individuals to access vital health data of stairlift users on a real-time basis, the present inventors have developed a modified version of a stairlift which allows for the remote monitoring of the health of these users by facilitating the measurement and transmittal of vital health data to a designated recipient such as, for example, a health professional, family member or carer.

### SUMMARY OF THE INVENTION

Thus, in accordance with a first aspect of the present invention there is provided a stairlift device which comprises passenger carrying means, guide means and transporting means, wherein said stairlift additionally comprises means for the detection of vital health data of passengers and transmittal of said data to remote locations.

Typically, the means for the detection of vital health data is located in the passenger carrying means.

In typical embodiments of the invention, the passenger carrying means comprises detectors for monitoring vital health indicators including, for example, heart rate, temperature, blood pressure, weight, and the like of a user located in the passenger carrying means.

Said detection means generally comprises non-intrusive detectors that do not require direct contact between the detector and the skin of the passenger.

In typical embodiments of the invention, the measured data collected when the user is located in the stairlift, is subsequently transmitted in real-time to a specified recipient, typically in a remote location. Transmission may be by means of a wired or wireless system.

In embodiments of the invention, the means for detection of vital health data may be used for passive, long term monitoring of passengers, so as to allow for the acquisition of data for trend analysis.

Embodiments of the invention envisage the use of biotelemetric systems for the detection and transmission of data. Typical biotelemetric systems may comprise
(a) at least one sensor appropriate for the particular signals to be monitored;
(b) at least one transmitter, which is typically electrically or battery powered;
(c) at least one radio antenna and receiver; and
(d) at least one display unit, optionally capable of concurrently presenting information from multiple patients.
In alternative embodiments of the invention, the radio antenna(e) and receiver(s) may be replaced by a hard-wired system.

Transmission systems may, for example, be selected from GSM, Bluetooth, SMS, 3G and 4G systems, and the like.

Typical sensors, transmitters, radio antennae and receivers and display units suitable for use in the apparatus of the invention may be selected from amongst those devices which would be well known and readily available to those skilled in the relevant area of technology.

As well as providing such data on a routine, real-time basis, the system may also be adapted so as to provide alerts where the data, such as heart rate, show significant abnormalities, such that that the life of the user may be at risk, thereby significantly improving response times, and allowing for immediate assistance and attention to be provided.

According to a further aspect of the present invention, there is provided a method for the detection of vital health data of passengers and transmittal of said data to remote locations, said method comprising:

(a) providing a stairlift device according to the first aspect of the invention;

(b) measuring health data by use of the means for the detection of said data; and

(c) transmitting said data to a remote location.

In the light of such data, health professionals in remote locations are able to monitor key vital health indicators such as heart rate, temperature, blood pressure, weight, and the like, of users of the stairlift device on a routine, real-time basis. In this way, they can be alerted to situations where data such as heart rate show significant abnormalities, so that improved response times can be achieved within which to provide the appropriate assistance and attention to the user.

A third aspect of the invention envisages the use of a stairlift device according to the first aspect of the invention for the detection of vital health data of passengers and transmittal of said data to remote locations.

The use of the stairlift device of the invention in this way is particularly applicable to the health monitoring of users who, typically, may be old or suffering from certain infirmities.

### BRIEF DESCRIPTION OF THE DRAWINGS

Embodiments of the invention are further described hereinafter with reference to the accompanying drawings, in which:

FIG. 1 shows a typical passenger carrying means, in the form of a seat, for use in the device of the invention.

### DESCRIPTION OF THE INVENTION

The invention provides a stairlift device which comprises passenger carrying means, guide means and transporting means, wherein said stairlift additionally comprises means for the detection of vital health data and transmittal of said data to remote locations.

The passenger carrying means is typically connected to the guide means by way of mounting means, attached to the passenger carrying means, which may be located within the guide means. Movement of the mounting means along the path of travel of the guide means thereby causes the passenger carrying means to be propelled to its desired destination, said movement being facilitated by the transporting means. Said transporting means allows for propulsion of the passenger carrying means, and preferably takes the form of an electric motor, this generally being powered electrically or by means of an independent battery. The transporting means may conveniently be located adjacent the passenger carrying means and is often integral with the mounting means.

Typically, the guide means comprises a close fitting channel into which said mounting means may be inserted, and which defines the path of travel of said passenger carrying means. Suitable guide means may, for example, comprise a metallic member in which a suitable channel or groove has been formed, the dimensions of said channel or groove being suitable to receive said mounting means. Typically, the guide means is provided in the form of a guide rail which is affixed to a staircase.

The passenger carrying means comprises any suitable means by which a passenger may be carried along the length of the guide means. Preferably, the passenger carrying means comprises seating means, wherein a person is seated for the purpose of ascending or descending the stairs; said seating means is typically provided in the form of a suitably designed chair. Alternatively, the passenger carrying means may comprise means for carrying a standing passenger, such as a platform or similar attachment.

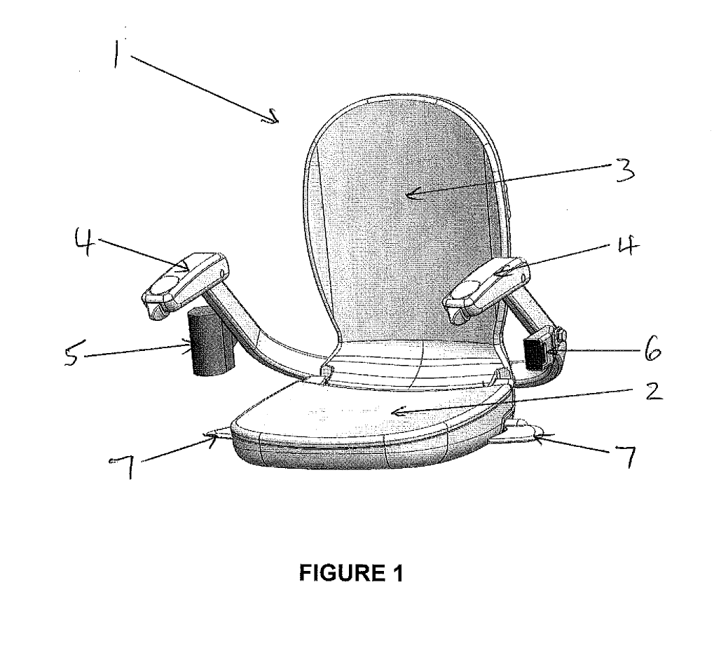

Referring to FIG. 1, there is illustrated a typical embodiment of seating means for use in the context of the stairlift device of the present invention in the form of a chair (1) which includes seat portion (2), back portion (3) and arms (4). The chair is also provided with seatbelt container (5) and clip (6) into which an end of the seatbelt is fixed after being drawn from container once a person is seated in the chair. The chair may conveniently be secured to the mounting means by the use of securing members (7) on the base of the chair. The means for the detection of vital health data may be located in any part of the chair, but are most conveniently located in the seat portion, back portion or arms. Transmittal of said data to remote locations may be effected either by means of a hard wired system, or by radio transmission.

According to certain embodiments of the invention, the guide means comprises substantially linear guide means. In many situations, however, it is the case that stairs to which stairlift devices are to be fitted are not entirely comprised in a simple linear arrangement, and flights of stairs often comprise some steps which are located at right angles to the remainder of the steps, thereby requiring a 90° turn to be included. Clearly, it is necessary to provide stairlift devices which are able to cater for such physical requirements, and which are capable of extending over the entire length of the staircase. The present invention includes such a facility, since a further embodiment allows for the provision of curved guide means which permit such geometries to be negotiated. Typically, the curved guide means could, for example, negotiate a sideward bend with a radius of 30.5-35.5 cm (12-14″) for a 1.3 cm (½″) pitch.

The stairlift device of the present invention has wide applicability. The vast majority of stairlift users are elderly and have mobility problems, therefore requiring an aid for negotiating stairs. In addition, many such users live alone, and have with varying degrees of health assistance and care provision. The present invention allows for the remote monitoring of their health, by sending vital health data to designated recipients, such as health professionals, carers and family members. The data collected and transmitted can be interpreted by the designated recipient(s), who are thereby able to monitor, for example, rises or falls in body temperature, blood pressure, weight, heart rate, and the like, in real time. The stairlift device may also be adapted and programmed such that it can send alerts to designated recipients when, for example, vital signs indicate that the condition of the user may be in danger of becoming critical or dangerous, thereby significantly improving emergency responses and overall health care.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A stairlift device which comprises passenger carrying means, guide means and transporting means, wherein said stairlift additionally comprises means for the detection of vital health data of passengers and transmittal of said data to remote locations.

2. A stairlift device as claimed in claim I wherein said means for the detection of vital health data is located in the passenger carrying means.

3. A stairlift device as claimed in claim 1 wherein said passenger carrying means comprises detectors for monitoring vital health indicators, wherein said vital health indicators optionally include heart rate, temperature, blood pressure and weight of a user located in the passenger carrying means.

4. (canceled)

5. A stairlift device as claimed in claim 1 wherein said means for the detection of vital health data comprises non-intrusive detectors that do not require direct contact between the detector and the skin of the passenger.

6. A stairlift device as claimed in claim 1 wherein data collected by the detectors is transmitted in real-time to a recipient in a remote location.

7. A stairlift device as claimed in claim 1 wherein transmission of data is by means of a wired or wireless system.

8. (canceled)

9. A stairlift device as claimed in claim 1 wherein biotelemetric systems are used for the detection and transmission of data, wherein said biotelemetric systems optionally comprise:
(a) at least one sensor appropriate for the particular signals to be monitored;
(b) at least one transmitter, which is optionally electrically or battery powered, and which is optionally selected from GSM, Bluetooth, SMS, 3G and 4G systems;
(c) at least one radio antenna and receiver or hard wired system; and
(d) at least one display unit, optionally capable of concurrently presenting information from multiple patients.

10. (canceled)

11. (canceled)

12. A stairlift device as claimed in claim 1 wherein said passenger carrying means is connected to the guide means by way of mounting means, wherein said mounting means is optionally attached to said passenger carrying means.

13. A stairlift device as claimed in claim 8 wherein said mounting means is located within said guide means.

14. A stairlift device as claimed in claim 1 wherein movement of the passenger carrying means along the path of travel of the guide means to its desired destination is facilitated by transporting means, wherein said transporting means optionally comprises an electric motor and said electric motor is optionally powered electrically or by means of an independent battery.

15. (canceled)

16. (canceled)

17. A stairlift device as claimed in claim 10 wherein said transporting means is located adjacent said passenger carrying means.

18. A stairlift device as claimed in claim 10 wherein said transporting means is integral with said mounting means.

19. A stairlift device as claimed in claim 1 wherein said guide means comprises a close fitting channel into which said mounting means is inserted, and which defines the path of travel of said passenger carrying means.

20. A stairlift device as claimed in claim 1 wherein said guide means comprises a metallic member in which a channel or groove is formed to receive said mounting means.

21. A stairlift device as claimed in claim 1 wherein said passenger carrying means comprises seating means, wherein said seating means optionally comprises a chair.

22. (canceled)

23. A stairlift device as claimed in claim 1 wherein said passenger carrying means comprises a platform.

24. A stairlift device as claimed in claim 1 wherein said guide means comprises substantially linear guide means.

25. A stairlift device as claimed in claim 1 which comprises curved guide means.

26. A method for the detection of vital health data of passengers and transmittal of said data to remote locations, said method comprising:
(a) providing a stairlift device as claimed in claim 1;
(b) measuring health data by use of the means for the detection of said data; and
(c) transmitting said data to a remote location,
wherein said method is optionally for the remote monitoring of vital health indicators.

27. (canceled)

28. The use of a stairlift device as claimed in claim 1 for the detection of vital health data to users and transmittal of said data to remote locations, wherein said use is for the health monitoring of users of the stairlift devices.

29. (canceled)
